(19) United States
(12) Patent Application Publication
(10) Pub. No.: US 2009/0048260 A1
(43) Pub. Date: Feb. 19, 2009

(54) Use of pyrrolopyrazine derivatives for the production of medicaments for the treatment of mucoviscidosis and diseases related to protein addressing errors in cells

(76) Inventors: Frederic Becq, Poitiers (FR); Laurent Meijer, Roscoff (FR); Yvette Mettey, Vincennes (FR)

Correspondence Address: WILMERHALE/BOSTON, 60 STATE STREET, BOSTON, MA, 02109, US

(21) Appl. No.: 11/665,294

(22) PCT Filed: Oct. 14, 2005
(86) PCT No.: PCT/FR05/02558

§ 371 Date: Apr. 13, 2007

(30) Foreign Application Priority Data
Oct. 15, 2004 (FR) 0410962

(51) Int. Cl. A61K 31/4985; A61P 11/00; A61P 1/18

(52) U.S. Cl. 514/249

(57) ABSTRACT

The invention relates to the use of pyrrolopyrazine derivatives for the treatment of mucoviscidosis and diseases related to protein addressing errors in cells, said derivatives being of formula (I): where R2 and R3 independently=H, C1-C6 straight or branched chain optionally-substituted alkyl, R6=an aromatic ring Ar or cycloalkyl, optionally substituted, said cycloalkyl optionally substituted by an aryl group which may equally be substituted, R7=H, C1-C6 alkyl, (alk.)ₙ-Hal, CH₂—CH═CH₂, CH₂-cycloalkyl, CH₂—Ar and Z=H or CH₃.

The invention relates to the use of pyrrolopyrazine derivatives for manufacturing medicaments capable of restoring the targeting of endoplasmic reticulum proteins to the plasma membranes. It relates most particularly to the treatment of cystic fibrosis.

Cystic fibrosis (CF: Cystic Fibrosis) is the lethal autosomal recessive genetic disease which is the most widespread in European and North American populations. The CF gene (7q31 locus) encodes the transmembrane protein called CFTR (Cystic Fibrosis Transmembrane Conductance Regulator). Mutations of the CF gene cause abnormal transport of water and electrolytes across the cell membranes of various organs such as the lungs, the sweat glands, the intestine and the exocrine pancreas. Although there are more than 1000 mutations of the CFTR protein, the most frequent mutation (70% of patients) is the deletion of a phenylalanine in the NBF1 domain at position 508 (delF508). The main cause of mortality of CF patients is linked to this deletion and leads to infections or to pulmonary insufficiency caused by an increase in the viscosity of mucous. This viscosity causes the obstruction of the respiratory tracts and promotes infections by opportunistic bacteria. Deterioration is furthermore observed at the digestive and in particular pancreatic level (patient with pancreatic insufficiency). The CFTR protein is a glycoprotein of 1480 amino acids, belonging to the superfamily of ABC membrane transporters. CFTR is a chloride channel located in the apical plasma membrane of the pulmonary epithelial cells in healthy individuals.

CFTR is responsible for the transepithelial transport of water and electrolytes and allows, in a healthy individual, moisturization of the pulmonary airways.

In CF patients, homozygots for the delF508 mutation, and more generally for the class II mutants (mutations producing a protein absent from the plasma membrane), this protein is absent from the plasma membranes because of the poor targeting of the protein which is retained in the endoplasmic reticulum (ER). The moisturization of the pulmonary airways is no longer functional in this case. The deletion of delF508 disrupts the folding of the NBF1 domain and prevents complete processing of the protein which is therefore degraded very early during its biosynthesis. However, if the delF508 protein reaches the membrane, it functions as a chloride channel.

One of the keys to the treatment of this disease therefore consists in a retargeting of delF508 to the plasma membrane of cells at the level of which the delF508 transport activity may be stimulated by physiological agonists.

Surprisingly, the inventors have demonstrated that derivatives which are in particular known for their antiproliferative effect were capable of activating the wild-type CFTR and the mutated forms and of causing membrane relocalization of the delF508-CFTR protein, thus restoring its transmembrane transport capacity. Generally, these derivatives are capable of restoring a defect in the targeting of proteins in cells.

Furthermore, these derivatives have the advantage of being highly safe.

The aim of the invention is therefore to provide a novel use of these derivatives for manufacturing medicaments for the treatment of cystic fibrosis and diseases linked to a defect in the targeting of proteins in cells.

The derivatives used in accordance with the invention correspond to formula (I):

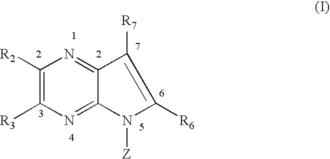

in which
R2 and R3 are identical or different and represent H, C1-C6 alkyl, said alkyl being a straight or branched alkyl chain, where appropriate substituted,
R6 is an aromatic ring Ar or a cycloalkyl, where appropriate substituted, said cycloalkyl being where appropriate substituted with an aryl group which may also be substituted,
R7 is H, C1-C6 alkyl, (alk.)ₙ-hal., CH₂—CH═CH₂, CH₂-cycloalkyl, CH₂—Ar,
Z is H or CH₃.

Preferably, R2 and R3, and/or Z and/or R7 are different from H.

Ar is preferably a phenyl, naphthyl, furyl, thienyl, pyridyl, cyclopropyl phenyl, phenyl dioxolyl.

“Cycloalkyl” is a C₃-C₆ cycloalkyl.

The substitutions of the alkyl group, of the aromatic or cycloalkyl ring are chosen from the group comprising one or more halogens (F, Cl, Br, I, CF₃), OH, NH₂, N(H, alkyl), N(alkyl)₂, O-alkyl, COOH, COO-alkyl, CONH₂, CON(H, alkyl), CON(alkyl)₂, NHCONH₂, NHCON(H, alkyl), NHCON(alkyl)₂, N(alkyl)CONH₂, N(alkyl)CON(H, alkyl), N(alkyl)CON(alkyl)₂, alkoxy, CN, O—SO₂—NH₂, O—SO₂N(H, alkyl), —O—SO₂—N(alkyl)₂, SH, S-alkyl. One or more substituents may be present.

“Alkyl” is a C1-C6 alkyl and comprises the isomers.

“Alkoxy” comprises a C1-C6 alkyl group.

“Alk.” is a C1-C6 alkylene group, n is 1-6, and “hal.” is F, Cl, Br, I or CF₃.

Said pyrrolo[2,3-b]pyrazines, also designated by aloisines below, are capable of restoring the targeting of the CFTR protein to the plasma membranes of cells and therefore constitute compounds of great interest for the treatment of diseases linked to such problems of targeting defects.

As illustrated by the examples, they are particularly effective for causing relocalization of the delF508-CFTR protein in cystic fibrosis where this protein is retained in the endoplasmic reticulum, and thus restoring its transmembrane transfer capacity.

Preferred pyrrolopyrazine derivatives have the formula (II):

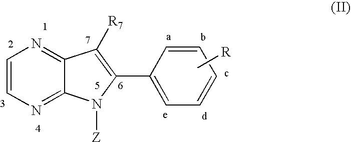

in which
the phenyl group at the 6-position is substituted with one, two or three substituents R chosen from the group comprising:
H, —OH, alkyl, —O alkyl, hal., —NH₂, —N(H, alkyl), —N(alkyl)₂, —O—SO₂—NH₂, —O—SO₂N(H, alkyl), —O—SO₂—N(alkyl)₂, —COOH, —COO-alkyl, CONH₂, —CON(H, alkyl), —CON (alkyl)₂,
R7 is H, alkyl, (alk.)ₙhal., —CH₂CH═CH₂, (alk.)ₙ-cycloalkyl, alk.-Ar, and
Z is H or CH₃.

In a preferred group, Z and/or R7 are different from H.

A compound most particularly preferred corresponds to aloisine A corresponding to formula (III)

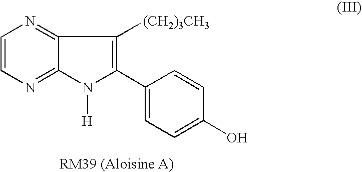

During the production of the medicaments, the active ingredients, used in therapeutically effective quantities, are mixed with the pharmaceutically acceptable vehicles for the mode of administration chosen. These vehicles may be solids or liquids.

Thus, for administration by the oral route, the medicaments prepared in the form of gelatin capsules, tablets, sugar-coated tablets, capsules, pills, drops, syrups and the like. Such medicaments may contain from 1 to 100 mg of active ingredient per unit.

For administration by injection (intravenous, subcutaneous, intramuscular), the medicaments are provided in the form of sterile or sterilizable solutions.

They may also be in the form of emulsions or suspensions.

The medicaments of the invention are more particularly administered in the form of aerosols.

The doses per dosage unit may vary from 1 to 50 mg of active ingredient. The daily dosage is chosen so as to obtain a final concentration of at most 100 μM as pyrrolopyrazine derivative in the blood of the treated patient.

Other characteristics and advantages of the invention will be given in the results reported below in order to illustrate the invention.

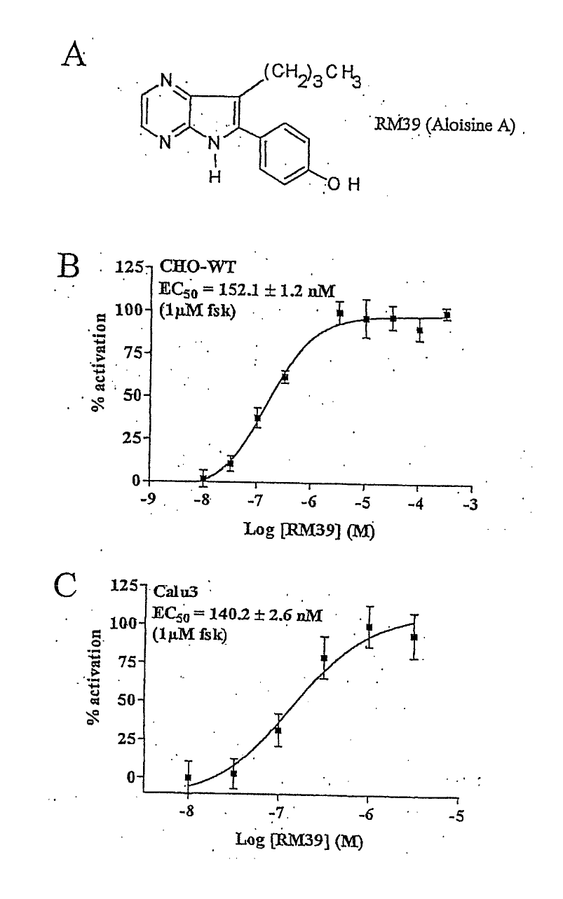

In these examples, reference is made to FIGS. 1 to 7 which represent, respectively:

FIGS. 1A to 1C, the formula of aloisine A, the activation of the CFTR chloride channel by aloisine A on CHO cells (FIG. 1B) and on human pulmonary epithelial cells Calu-3 (FIG. 1C);

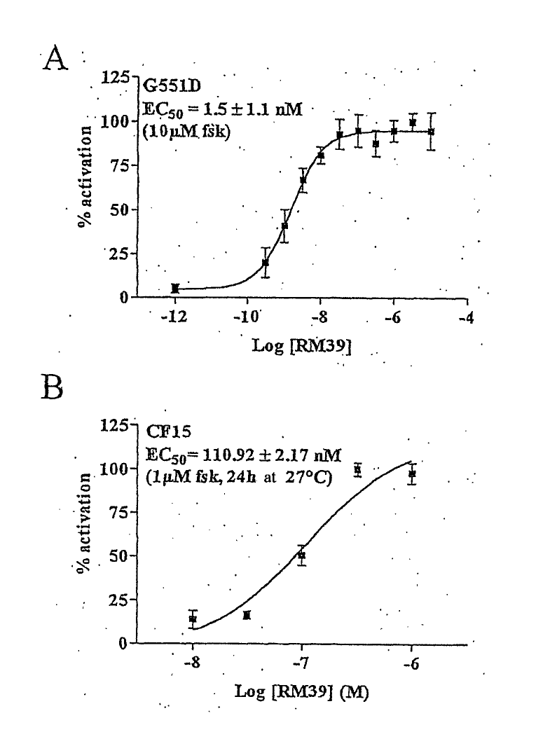

FIGS. 2A and 2B, the activation by aloisine A of the G551D-CFTR protein in CHO cells (FIG. 2A) and the delF508 protein in the pulmonary human epithelial cells of the CF15 line (FIG. 2B);

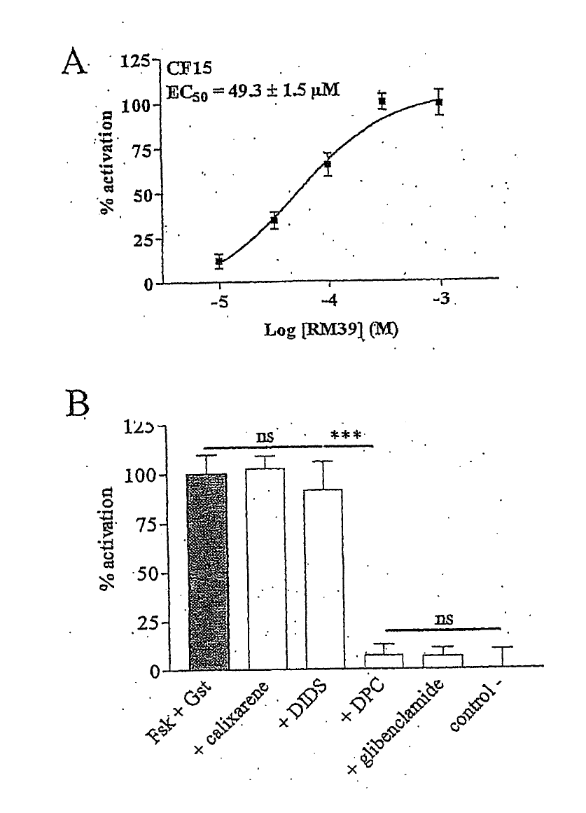

FIGS. 3A and 3B, the EC₅₀ of aloisine A at 50 μM (FIG. 3A) and the inhibitory effect of CTFR on the activity of delF508 after treatment with aloisine A;

FIG. 4, the localization of delF508 in CF patients and its retargeting to the membrane after treatment with aloisine A;

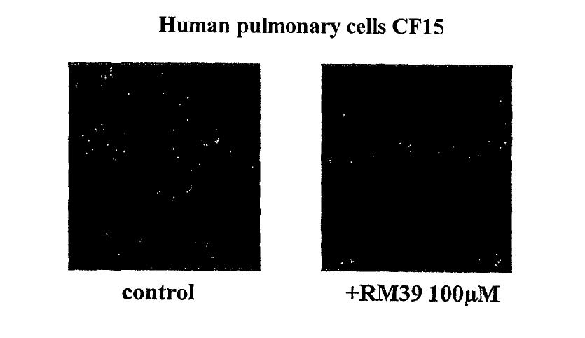

FIGS. 5A and 5B, tests of toxicity of aloisine A on CHO-WT cells after incubation for 2 h (FIG. 5A) and for 24 h (FIG. 5B),

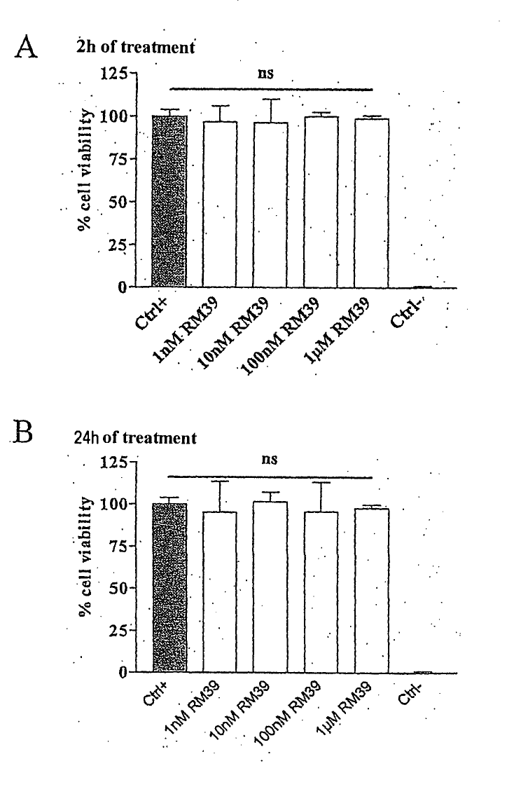

FIGS. 6A to 6C, immunolocalization of delF508-CFTR after 2 h of treatment with aloisine A,

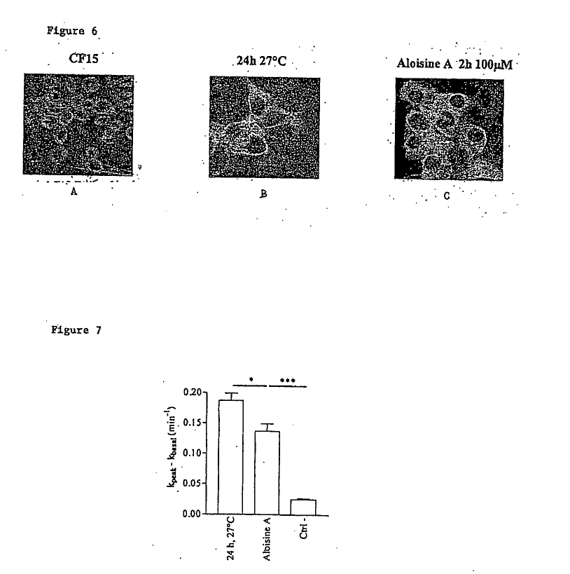

FIG. 7, the activation of delF508-CFTR in the CF15 cells after treatment with aloisine A.

### Materials and Methods

### M1. Cell Culture

CHO-WT cells: The CHO (Chinese Hamster Ovary) cells are fibroblasts which have been transfected with the wild-type CFTR (CFTR-WT) gene. These cells will therefore overexpress the CFTR protein.

Culture medium: MEM alpha medium (GIBCO)+7% fetal calf serum+0.5% penicillin/streptomycin+100 μM methotrexate (amethopterin, Sigma).

CF15 cells: CF15 cells are human epithelial cells of nasal origin which express the Δ508-CFTR gene.

Culture medium: DMEM medium+HAM F12+10% FCS+0.6% penicillin/streptomycin+growth factors (5 μg/ml insulin, 5 μg/ml transferrin, 5.5 μM epinephrine, 0.18 mM adenine, 10 ng/ml EGF, 2 nM T3, 1.1 μM hydrocortisone).

Calu-3 cells: Calu-3 cells are human epithelial cells of pulmonary origin which express the wild-type CFTR gene.

Culture medium: DMEM/F12 medium with glutamax+7% fetal calf serum+1% penicillin/streptomycin.

### M2. Immunolabeling

Immunolabeling makes it possible to reveal the cellular location of the CFTR protein using a primary anti-CFTR antibody (Ab), and then a secondary antibody anti-primary antibody labeled with the fluorophore Cy3.

The cells are inoculated beforehand on cover glass in appropriate culture medium. 3 washes with TBS (NaCl: 157 mM, Tris base: 20 μM, pH 7.4) of 5 min each are performed. The cells are then fixed by addition of TBS-paraformaldehyde (3%) for 20 min. After 3 washes with TBS (5 min), the cells are incubated with TBS-triton 0.1% (10 min) which allows the formation of holes in the cell membrane and then 3 washes with TBS are again carried out before bringing the cells into contact with TBS-BSA 0.5%-saponin 0.05% for 1 h. The cells are then incubated with the anti-C-terminal CFTR primary antibody (2 μg/ml) for 1 h. 3 washes with TBS-BSA-saponin of 5 min each are carried out before the incubation with the secondary antibody GAM-cy3 (1/400) for 1 h. Following 2 washes with TBS of 5 min, the nuclei are labeled by incubating with Topro3 (1/1000) for 5 min. Next, the cover glass may be mounted on the slide after 3 final washes with TBS of 5 min. The slides are examined under a confocal microscope (Bio-Rad) using a laser excitation at the appropriate wavelengths. In order to distinguish between the Cy3 and Topro3 labeling, the fluorescence color of Topro3 was changed to blue (color of the nuclei).

M3. Radiotracer efflux

the measurements of chloride ion transport in the cells was carried out with the aid of the radioactive iodide efflux technique (Becq et al., 1999; Dormer et al., 2001). The tracer (¹²⁵I) is incorporated into the intracellular medium. Next, the quantity of radiotracer which leaves the cell is counted after the addition of various pharmacological agents. Iodide is used as a tracer for the transport of chloride ions. ¹²⁵I further has the advantage of having a short life compared with that of other markers such as ³⁵Cl (respective ½ life: 30 days and 300000 years).

The cells are cultured on 24-well plates in a suitable medium. 2 rinses with efflux medium (NaCl: 136.6 mM, KCl: 5.4 mM, KH₂PO₄: 0.3 mM, NaH₂PO₄: 0.3 mM, NaHCO₃: 4.2 mM, CaCl₂: 1.3 mM, MgCl₂: 0.5 mm, MgSO₄: 0.4 mM, HEPES: 10 mM, D-glucose: 5.6 mM) are carried out in order to remove the dead cells which release the radioactivity anarchically. The cells are then incubated with 500 μl of load (1 μCi/ml of ¹²⁵INa) for 30 min for CHO-WT or 1 h for CF15 and Calu-3. The iodide equilibrates on either side of the cell membrane. A robot (MultiPROBE, Packard) carries out the following steps: the loading medium is rinsed with efflux medium in order to remove the extracellular radioactivity. The supernatant is collected every minute in hemolysis tubes and the medium is replaced with an equivalent volume (500 μl). The samples collected for the first 3 minutes are not supplemented with drug, they make it possible to obtain a stable baseline, characterizing the passive outflow of the I ions. The next 7 samples are obtained in the presence of the molecule to be tested. At the end of the experiment, the cells are lyzed by adding 500 μl of NaOH (0.1 N)/0.1% SDS (30 min), thus, the radioactivity which remained inside the cell may be determined. The radioactivity present in the hemolysis tubes is counted as counts per minute (cpm) using a gamma counter (Cobra II, Packard). The results in cpm are expressed in the form of rate of radioactive iodide outflow (R) according to the following formula:

R (min⁻¹)=[In(¹²⁵I t₁)−In(¹²⁵I t₂)]/(t₁−t₂) with ¹²⁵I t₁: cpm at time t₁; ¹²⁵I t₂: cpm at time t₂. This iodide flow is represented in the form of a curve. In order to quantify the outflow of iodide due to the administration of the molecule tested, the following relative flow is calculated which makes it possible to dispense with the basic flow: relative rate (min⁻¹)=Rpeak−Rbasal. Finally, these results are normalized in order to be able to compare the effect of the various drugs with each other. The results are presented in the form of a mean+/−SEM. The student's statistical test is used to compare the effect of the drugs to the controls (the values corresponding to P<0.01 are considered as statistically significant).

### M4. Cytotoxicity Test

The test of toxicity to MTT is a calorimetric test which is based on capacity of mitochondrial dehydrogenases to metabolize MTT (yellow tetrazolium salt) to formazan (purple). The absorbance, which is proportional to the concentration of dye converted, can then be measured by spectrophotometry. The cells are incubated on 96-well plates in the presence of the agent to be tested for 2 h. 3 controls are prepared: 100% living cells: cells without agent; 0% living cells: cells left in the open air; blank: medium without cells. The cells are rinsed with RPMI medium without phenol red so that the color of the medium does not interfere with the measurements of absorbance. Next, they are incubated for 4 h with 100 μl of RPMI solution supplemented with MTT (0.5 mg/ml). The medium is then removed, the addition of 100 μl of DMSO makes it possible to solubilize the dye converted (formazan). The absorbance is measured by spectrophotometry at 570 nm (purple); 630 nm (background). In order to eliminate the background, the following calculation is carried out: DOᵣₑₐₗ=DO₅₇₀ₙₘ−DO₆₃₀ₙₘ. Next, the results are normalized with respect to the controls (100% and 0% of living cells) and are presented in the form of mean+/−SEM.

### Results

R1. Aloisine A Activates Wild-Type (WT) CFTR, G551D and delF508

A first series of experiments shows that aloisine A is capable of activating the CFTR chloride channel in CHO cells (EC₅₀=152.1 nM, FIG. 1A) and in the human pulmonary epithelial cell line Calu-3 (EC₅₀=140.1 nM, FIG. 1B).

Two mutated forms of CFTR were also tested. They are the mutant G551D and delF508. FIG. 2 shows that aloisine A activates the protein G551D-CFTR in the CHO cell (EC₅₀=1.5 nM, FIG. 2A) and the protein delF508 (EC₅₀=110 nM, FIG. 2B) in the pulmonary human epithelial cell line CF15 after incubation of the cells at 27° C. (procedure which makes it possible to relocalize the protein delF508 to the membrane).

These results demonstrate that this molecule is an activator of wild-type CFTR, G551D and delF508.

R2. Effect of Aloisine A on the Targeting of delF508 in CF15 cells

The study of the targeting of the delF508-CFTR protein was carried out by combining pharmacological and cell imaging approaches, and biochemical and electrophysiological tests on pulmonary human epithelial cells CF15 homozygous for the delF508 deletion.

For each experiment, the addition of a cocktail (10 μM forskolin+30 μM genistein) allows the activation of CFTR when it is attached to the membrane. Thus, an iodide efflux can be observed if the targeting of delF508 has been restored.

The results, presented in the form of a histogram, were normalized with respect to a reference treatment (treatment of the cells with 250 μM MPB-91 for 2 h) for which it is considered that the CFTR activity is 100%.

FIG. 3A shows that after a 2 h treatment with 100 μM of aloisine A, the delF508-CFTR activity is restored.

These results thus demonstrate that the treatment of the CF15 cells with aloisine A for 2 h at 37° C. restores targeting of the delF508 protein and allows it to function as an ion transporter.

In the absence of treatment of the cells, the delF508 protein is not a membrane protein and there is no iodide efflux stimulated by the cocktail (10 μM forskolin+30 μM genistein). The EC₅₀ (concentration of the molecule which gives 50% maximum efficacy) of aloisine A was determined at 50 μM (FIG. 3A, n=4, for each condition). To define the observed effect precisely, known inhibitors of CFTR on the activity of delF508 were tested after treatment with aloisine A. The results presented in FIG. 3B show that this transport is blocked by glibenclamide and DPC but insensitive to DIDS and to calixarene. This pharmacological profile corresponds to that of CFTR.

In the CF patients, the delF508 protein is absent from the plasma membranes because of a poor targeting of the protein which is retained in the endoplasmic reticulum (ER). By cell imaging in CF15 cells, delF508 is indeed localized in intracellular compartments (FIG. 4). On the other hand, treatment with 100 μM aloisine A makes it possible to redirect the delF508 protein to the membrane as shown in FIG. 4.

The immunolocalization of delF508-CFTR in CF15 cells is also represented in FIGS. 6A to 6C which show, respectively, FIG. 6A: the confocal visualization of CFTR-delF508 in CF15 cells with a mouse anti-CFTR monoclonal antibody; FIG. 6B: the CF15 cells treated for 24 h at 27° C., used as positive control; FIG. 6C: the CF15 cells treated for 2 h with aloisine A (100 μM).

The activation of delF508-CFTR in the CF15 cells after treatment with aloisine A is represented in FIG. 7.

The iodide effluxes were observed after 2 h of incubation with these compounds or in the absence of treatment.

The CF15 cells which have been subjected to a 24 h treatment at 27° C. were used as positive control and the untreated CF15 cells as negative control (37° C.).

The table below shows a summary of competition experiments carried out by the iodide efflux technique between aloisine A and the ER chaperone machinery.

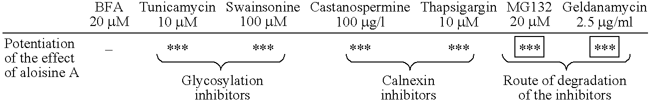

— inhibition, **P < 0.01 (student's t-test)

An inhibition is observed of the effect of aloisine A by Brefeldin A (BFA), an inhibitor of the ERGIC vesicular traffic, which shows that aloisine A induces retargeting of the delF508-CFTR protein.

### R3. Cytotoxicity of Aloisine A

With the aim of testing the cytotoxicity of aloisine A, CHO-WT cells were incubated for 2 h (FIG. 5A) or 24 h (FIG. 5B) with various concentrations of aloisine A before being subjected to the MTT cell viability test. The results show that the cells are viable for all the concentrations. This molecule therefore does not exhibit cell cytotoxicity.

Iodide efflux, patch clamp and Ussing chamber experiments have shown that aloisine A is an activator of the wild-type and mutated CFTR proteins (F508del, G551D, G1349D). This high-affinity molecule exhibits EC₅₀ values varying from 1.5 to 303 nM according to the mutant tested.

Furthermore, the experiments carried out have demonstrated that a treatment of CF15 cells (F508del/F508del) with aloisine A allowed retargeting of the mutated protein F508del CFTR to the membrane.

Iodide efflux experiments have shown that aloisine A allows retargeting of the F508del-CFTR protein after 2 h of treatment with an EC₅₀ of 49 μM.

These results demonstrate that aloisine A is an activator of CFTR at low concentration but that this molecule can also act as a pharmacological chaperone at high concentration.

Furthermore, studies of aloisine A toxicity, carried out on animals, have revealed a very low toxicity.

The results above were also verified with other pyrrolopyrazine derivatives.

### EXAMPLE OF FORMULATION

A solution for inhalation is prepared with a vial nebulizer from sodium chloride, dehydrated calcium chloride and water for injection.

Aloisine A is added as active ingredient.

The solution is formulated in 2.5 ml vials.

Vials containing 5, 10 mg or 20 mg of aloisine are thus prepared.

### BIBLIOGRAPHIC REFERENCES

BECQ et al. (1999) Journal of Biological Chemistry 274, 27415-27425.
DORMER et al. (2001) Journal of Cell Science 114, 4073-4081.

1-9. (canceled)

10. A method for treating cystic fibrosis in a patient comprising administering to a patient in need of such treatment an effective amount of a compound of formula (I): 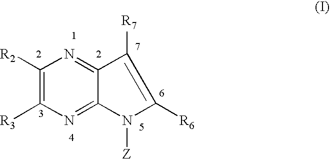
in which
—R₂ and R₃ are identical or different and represent H or a (C₁-C₆)-alkyl, said alkyl being a straight or branched alkyl chain, which is optionally substituted;
R₆ is an aromatic ring (Ar) or a cycloalkyl, wherein said aromatic ring or cycloalkyl is optionally substituted;
R₇ is H, a (C₁-C₆)-alkyl, a [(C₁-C₆)-alkylene]ₙ—X wherein X is a member selected from the group consisting of F, Cl, Br, I or CF₃, CH₂—CH═CH₂ and n is a number from 1 to 6, CH₂-cycloalkyl, or CH₂—Y wherein Y is an aromatic ring; and
Z is H or CH₃;
wherein the optional substitution for R₂, R₃ and R₆ is selected from the group consisting of F, Cl, Br, I, CF₃, OH, NH₂, N(H—(C₁-C₆)-alkyl), N((C₁-C₆)-alkyl)₂, O—(C₁-C₆)-alkyl, COOH, COO—(C₁-C₆)-alkyl, CONH₂, CON(H—(C₁-C₆)-alkyl), CON((C₁-C₆)-alkyl)₂, NHCONH₂, NHCON(H—(C₁-C₆)-alkyl), NHCON((C₁-C₆)-alkyl)₂, N((C₁-C₆)-alkyl)CONH₂, N((C₁-C₆)-alkyl)CON(H—(C₁-C₆)-alkyl), N((C₁-C₆)-alkyl)CON((C₁-C₆)-alkyl)₂, (C₁-C₆)-alkoxy, CN, O—SO₂—NH₂, O—SO₂N(H—(C₁-C₆)-alkyl), —O—SO₂—N((C₁-C₆)-alkyl)₂, SH, and S—(C₁-C₆)-alkyl.

11. The method according to claim 10, wherein at least one of R₂, R₃, Z and R₇ is different from H.

12. The method according to claim 10, wherein at least one of R₂ and R₃ is an alkyl group that is substituted.

13. The method according to claim 10, wherein R₆ is an alkyl, aromatic or cycloalkyl group that is substituted.

14. The method according to claim 13, wherein R₆ is a cycloalkyl that is substituted with an aryl group.

15. The method according to claim 14, wherein R₆ is a cycloalkyl that is substituted with an aryl group that is substituted with at least one member selected from the group consisting of F, Cl, Br, I, CF₃, OH, NH₂, N(H—(C₁-C₆)-alkyl), N((C₁-C₆)-alkyl)₂, O—(C₁-C₆)-alkyl, COOH, COO—(C₁-C₆)-alkyl, CONH₂, CON(H—(C₁-C₆)-alkyl), CON((C₁-C₆)-alkyl)₂, NHCONH₂, NHCON(H—(C₁-C₆)-alkyl), NHCON((C₁-C₆)-alkyl)₂, N((C₁-C₆)-alkyl)CONH₂, N((C₁-C₆)-alkyl)CON(H—(C₁-C₆)-alkyl), N((C₁-C₆)-alkyl)CON((C₁-C₆)-alkyl)₂, (C₁-C₆)-alkoxy, CN, O—SO₂—NH₂, O—SO₂N(H—(C₁-C₆)-alkyl), —O—SO₂—N((C₁-C₆)-alkyl)₂, SH, and S—(C₁-C₆)-alkyl.

16. A method for treating cystic fibrosis in a patient comprising administering to a patient in need of such treatment an effective amount of a compound of formula (II): 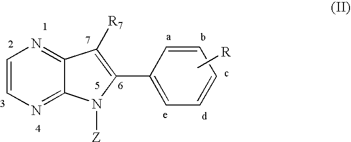
in which
phenyl group at the 6-position is substituted with one, two or three substituents R selected from the group consisting of H, —OH, —(C₁-C₆)-alkyl, —O—(C₁-C₆)-alkyl, F, Cl, Br, I or CF₃, —NH₂, —N(H—(C₁-C₆)-alkyl, —N((C₁-C₆)-alkyl)₂, —O—SO₂—NH₂, —O—SO₂N(H—(C₁-C₆)-alkyl), —O—SO₂—N((C₁-C₆)-alkyl)₂, —COOH, —COO—(C₁-C₆)-alkyl, CONH₂, —CON(H—(C₁-C₆)-alkyl), and —CON((C₁-C₆)-alkyl)₂;
R₇ is H, —(C₁-C₆)-alkyl, —CH₂CH═CH₂, ((C₁-C₆)-alkyl)ₙ-cycloalkyl, [(C₁-C₆)-alkylene]ₙ —X wherein X is a member selected from the group consisting of F, Cl, Br, I or CF₃, and n is a number from 1 to 6; and
Z is H or CH₃,
wherein the optional substitution for R₂, R₃ and R₆ is selected from the group consisting of F, Cl, Br, I, CF₃, OH, NH₂, N(H—(C₁-C₆)-alkyl), N((C₁-C₆)-alkyl)₂, O—(C₁-C₆)-alkyl, COOH, COO—(C₁-C₆)-alkyl, CONH₂, CON(H—(C₁-C₆)-alkyl), CON((C₁-C₆)-alkyl)₂, NHCONH₂, NHCON(H—(C₁-C₆)-alkyl), NHCON((C₁-C₆)-alkyl)₂, N((C₁-C₆)-alkyl)CONH₂, N((C₁-C₆)-alkyl)CON(H—(C₁-C₆)-alkyl), N((C₁-C₆)-alkyl)CON((C₁-C₆)-alkyl)₂, (C₁-C₆)-alkoxy, CN, O—SO₂—NH₂, O—SO₂N(H—(C₁-C₆)-alkyl), —O—SO₂—N((C₁-C₆)-alkyl)₂, SH, and S—(C₁-C₆)-alkyl.

17. The method according to claim 16, wherein Z and/or R₇ are different from H.

18. A method for treating cystic fibrosis in a patient comprising administering to a patient in need of such treatment an effective amount of a compound of formula (III)

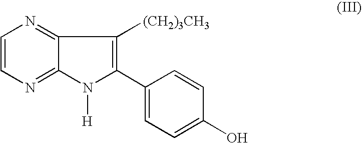

19. The method according to claim 10 wherein the compound is orally administered in a form of a gelatin capsule, tablet, sugar-coated tablet or capsule.

20. The method according to claim 10 wherein the compound is administered by injection, in the form of a solution.

21. The method according to claim 10 wherein the compound is administered in aerosol form.

22. A method for treating diseases linked to a defect of protein targeting in cells in a patient comprising administering to a patient in need of such treatment a compound of formula (I): 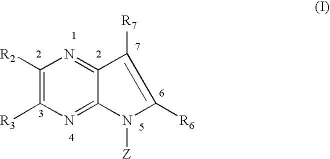
in which
—R₂ and R₃ are identical or different and represent H or a (C₁-C₆)-alkyl, said alkyl being a straight or branched alkyl chain, which is optionally substituted;
R₆ is an aromatic ring (Ar) or a cycloalkyl, wherein said aromatic ring or cycloalkyl is optionally substituted;
R₇ is H, a (C₁-C₆)-alkyl, a [(C₁-C₆)-alkylene]ₙ—X wherein X is a member selected from the group consisting of F, Cl, Br, I or CF₃, CH₂—CH═CH₂ and n is a number from 1 to 6, CH₂-cycloalkyl, or CH₂—Y wherein Y is an aromatic ring; and
Z is H or CH₃;
wherein the optional substitution for R₂, R₃ and R₆ is selected from the group consisting of F, Cl, Br, I, CF₃, OH, NH₂, N(H—(C₁-C₆)-alkyl), N((C₁-C₆)-alkyl)₂, O—(C₁-C₆)-alkyl, COOH, COO—(C₁-C₆)-alkyl, CONH₂, CON(H—(C₁-C₆)-alkyl), CON((C₁-C₆)-alkyl)₂, NHCONH₂, NHCON(H—(C₁-C₆)-alkyl), NHCON((C₁-C₆)-alkyl)₂, N((C₁-C₆)-alkyl)CONH₂, N((C₁-C₆)-alkyl)CON(H—(C₁-C₆)-alkyl), N((C₁-C₆)-alkyl)CON((C₁-C₆)-alkyl)₂, (C₁-C₆)-alkoxy, CN, O—SO₂—NH₂, O—SO₂N(H—(C₁-C₆)-alkyl), —O—SO₂—N((C₁-C₆)-alkyl)₂, SH, and S—(C₁-C₆)-alkyl.

23. The method according to claim 22, wherein at least one of R₂, R₃, Z and R₇ is different from H.

24. The method according to claim 22, wherein at least one of R₂ and R₃ is an alkyl group that is substituted.

25. The method according to claim 22, wherein R₆ is an alkyl, aromatic or cycloalkyl group that is substituted.

26. The method according to claim 25, wherein R₆ is a cycloalkyl that is substituted with an aryl group.

27. The method according to claim 26, wherein R₆ is a cycloalkyl that is substituted with an aryl group that is substituted with at least one member selected from the group consisting of F, Cl, Br, I, CF₃, OH, NH₂, N(H—(C₁-C₆)-alkyl), N((C₁-C₆)-alkyl)₂, O—(C₁-C₆)-alkyl, COOH, COO—(C₁-C₆)-alkyl, CONH₂, CON(H—(C₁-C₆)-alkyl), CON((C₁-C₆)-alkyl)₂, NHCONH₂, NHCON(H—(C₁-C₆)-alkyl), NHCON((C₁-C₆)-alkyl)₂, N((C₁-C₆)-alkyl)CONH₂, N((C₁-C₆)-alkyl)CON(H—(C₁-C₆)-alkyl), N((C₁-C₆)-alkyl)CON((C₁-C₆)-alkyl)₂, (C₁-C₆)-alkoxy, CN, O—SO₂—NH₂, O—SO₂N(H—(C₁-C₆)-alkyl), —O—SO₂—N((C₁-C₆)-alkyl)₂, SH, and S—(C₁-C₆)-alkyl.

28. A method for treating diseases linked to a defect of protein targeting in cells in a patient comprising administering to a patient in need of such treatment an effective amount of a compound of formula (II): 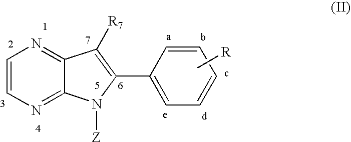
in which
phenyl group at the 6-position is substituted with one, two or three substituents R selected from the group consisting of H, —OH, —(C₁-C₆)-alkyl, —O—(C₁-C₆)-alkyl, F, Cl, Br, I or CF₃, —NH₂, —N(H—(C₁-C₆)-alkyl, —N((C₁-C₆)-alkyl)₂, —O—SO₂—NH₂, —O—SO₂N(H—(C₁-C₆)-alkyl), —O—SO₂—N((C₁-C₆)-alkyl)₂, —COOH, —COO—(C₁-C₆)-alkyl, CONH₂, —CON(H—(C₁-C₆)-alkyl), —CON((C₁-C₆)-alkyl)₂;
R₇ is H, —(C₁-C₆)-alkyl, —CH₂CH═CH₂, ((C₁-C₆)-alkyl)ₙ-cycloalkyl, [(C₁-C₆)-alkylene]ₙ—X wherein X is a member selected from the group consisting of F, Cl, Br, I or CF₃, and n is a number from 1 to 6; and
Z is H or CH₃.

29. The method according to claim 28, wherein Z and/or R₇ are different from H.

30. A method for treating diseases linked to a defect of protein targeting in cells in a patient comprising administering to a patient in need of such treatment an effective amount of a compound of formula (III)

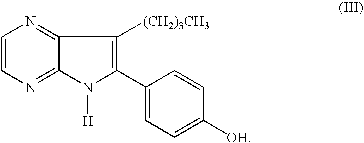

31. The method according to claim 22 wherein the compound is orally administered in a form of a gelatin capsule, tablet, sugar-coated tablet or capsule.

32. The method according to claim 22 wherein the compound is administered by injection, in the form of a solution.

33. The method according to claim 22 wherein the compound is administered in aerosol form.

34. A pharmaceutical composition of matter comprising a compound of formula (I) in an amount effective for treating cystic fibrosis in a patient, wherein formula (I) is: 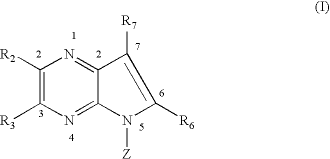
in which
R₂ and R₃ are identical or different and represent H or a (C₁-C₆)-alkyl, said alkyl being a straight or branched alkyl chain, which is optionally substituted;
R₆ is an aromatic ring (Ar) or a cycloalkyl, wherein said aromatic ring or cycloalkyl is optionally substituted;
R₇ is H, a (C₁-C₆)-alkyl, a [(C₁-C₆)-alkylene]ₙ—X wherein X is a member selected from the group consisting of F, Cl, Br, I or CF₃, CH₂—CH═CH₂ and n is a number from 1 to 6, CH₂-cycloalkyl, or CH₂—Y wherein Y is an aromatic ring; and
Z is H or CH₃,
wherein the optional substitution for R₂, R₃ and R₆ is selected from the group consisting of F, Cl, Br, I, CF₃, OH, NH₂, N(H—(C₁-C₆)-alkyl), N((C₁-C₆)-alkyl)₂, O—(C₁-C₆)-alkyl, COOH, COO—(C₁-C₆)-alkyl, CONH₂, CON(H—(C₁-C₆)-alkyl), CON((C₁-C₆)-alkyl)₂, NHCONH₂, NHCON(H—(C₁-C₆)-alkyl), NHCON((C₁-C₆)-alkyl)₂, N((C₁-C₆)-alkyl)CONH₂, N((C₁-C₆)-alkyl)CON(H—(C₁-C₆)-alkyl), N((C₁-C₆)-alkyl)CON((C₁-C₆)-alkyl)₂, (C₁-C₆)-alkoxy, CN, O—SO₂—NH₂, O—SO₂N(H—(C₁-C₆)-alkyl), —O—SO₂—N((C₁-C₆)-alkyl)₂, SH, and S—(C₁-C₆)-alkyl,
and a pharmaceutically acceptable carrier.

35. A pharmaceutical composition of matter comprising a compound of formula (II) in an amount effective for treating cystic fibrosis in a patient, wherein formula (II) is: 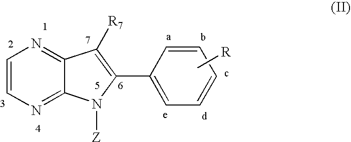
in which
phenyl group at the 6-position is substituted with one, two or three substituents R selected from the group consisting of H, —OH, —(C₁-C₆)-alkyl, —O—(C₁-C₆)-alkyl, F, Cl, Br, I or CF₃, —NH₂, —N(H—(C₁-C₆)-alkyl, —N((C₁-C₆)-alkyl)₂, —O—SO₂—NH₂, —O—SO₂N(H—(C₁-C₆)-alkyl), —O—SO₂—N((C₁-C₆)-alkyl)₂, —COOH, —COO—(C₁-C₆)-alkyl, CONH₂, —CON(H—(C₁-C₆)-alkyl), —CON((C₁-C₆)-alkyl)₂;
R₇ is H, —(C₁-C₆)-alkyl, —CH₂CH═CH₂, ((C₁-C₆)-alkyl)ₙ-cycloalkyl, [(C₁-C₆)-alkylene]ₙ—X wherein X is a member selected from the group consisting of F, Cl, Br, I or CF₃, and n is a number from 1 to 6; and
Z is H or CH₃,
and a pharmaceutically acceptable carrier.

36. A pharmaceutical composition of matter comprising a compound of formula (III) in an amount effective for treating cystic fibrosis in a patient, wherein formula (III) is:

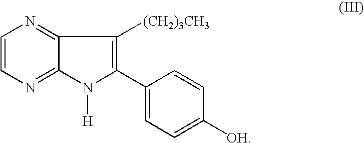

37. The pharmaceutical composition according to claim 34, wherein the composition is in an oral dosage form of a gelatin capsule, tablet, sugar-coated tablet or capsule.

38. The pharmaceutical composition according to claim 34, wherein the composition is in a form of a solution suitable for injection.

39. The pharmaceutical composition according to claim 34, wherein the composition is in an aerosol form.

40. The pharmaceutical composition according to claim 34 comprising 1 to 100 mg of the compound of formula (I).
